(19) United States
(12) Patent Application Publication
(10) Pub. No.: US 2026/0061111 A1
(43) Pub. Date: Mar. 5, 2026

(54) ORONASAL CONJUNCTIVAL SPRAY

(71) Applicant: Sheikh Parviz, Bethesda, MD (US)

(72) Inventor: Sheikh Parviz, Bethesda, MD (US)

(21) Appl. No.: 19/311,889

(22) Filed: Aug. 27, 2025

Related U.S. Application Data
(60) Provisional application No. 63/687,318, filed on Aug. 27, 2024.

(51) Int. Cl. A61M 3/02

(52) U.S. Cl. CPC A61M 3/0254

(57) ABSTRACT

A oronasal conjunctival spray may include a bottle for fluid and an applicator that may be screwable to the bottle. The spray applicator may include a nasal spray port for spraying fluid into a human nose, a conjunctival spray port for spraying fluid into a human eye, and an oral spray port for spraying fluid into a human oral cavity (mouth).

### FIELD OF THE INVENTION

The present disclosure relates generally to a spray, and more particularly to a oronasal conjunctival spray.

### BACKGROUND

Viral Upper Respiratory Tract Infections (e.g., COVID-19 and flu) can be highly contagious. Preventing the spread and early treatment of diseases is highly important. The respiratory epithelium, where the viruses enter and replicate in the mouth, nose, and conjunctiva. Nasal sprays deliver medications only through the nose to treat the nasal part of the disease and symptoms associated with it, such as nasal congestion and rhinorrhea, thereby leaving the user susceptible to infections from other viral entry points, as well as replication of the virus in the remaining parts (e.g., eyes and mouth)

### BRIEF OVERVIEW

The present disclosure is directed to a spray applicator including a nasal spray port for spraying fluid into a human nose and a conjunctival spray port for spraying fluid into a human eye and an oral spray port for spraying fluid into the oral cavity. The spray applicator may further include a pivot to redirect fluid to one of the nasal spray port, conjunctival spray port, or oral spray port. The spray applicator may further include one or more orifices to adjust an amount of fluid exiting one or more of the nasal spray port, conjunctival spray port, or oral spray port. The spray applicator may further include a first and second part, wherein the first part is movable against the second part, such that a movement of the first part against the second part sprays fluid from one or more of the nasal spray port, conjunctival spray port, or oral spray port. The spray applicator may further include a spring that pushes the first and second part away from each other. The spray applicator may further include a lock that locks the first part against the second part at a maximum distance.

The present disclosure is directed to a oronasal conjunctival spray, including a bottle for fluid and a spray applicator including a nasal spray port for spraying fluid into a human nose and a conjunctival spray port for spraying fluid into a human eye, wherein the spray applicator is screwable to the bottle. The oronasal conjunctival spray may further comprise a screw that is loosely attached to the spray applicator. The spray applicator may include a hosepipe.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical items.

FIG. 1 illustrates a bottle and an applicator of an oronasal conjunctival spray, in accordance with one or more embodiments of the present disclosure.

FIG. 2 illustrates an applicator of the oronasal conjunctival spray, in accordance with one or more embodiments of the present disclosure.

FIG. 3A illustrates the oronasal conjunctival spray spraying fluid into a human mouth, in accordance with one or more embodiments of the present disclosure.

FIG. 3B illustrates the oronasal conjunctival spray spraying fluid into a human nose, in accordance with one or more embodiments of the present disclosure.

FIG. 3C illustrates the oronasal conjunctival spray spraying fluid into a human eye, in accordance with one or more embodiments of the present disclosure.

### DETAILED DESCRIPTION

In the following detailed description, references are made to the accompanying drawings that form a part hereof, and which are shown by way of illustration specific embodiments or examples. Referring now to the drawings, in which like numerals represent like elements through the several figures, aspects of a finger contact point trainer will be described.

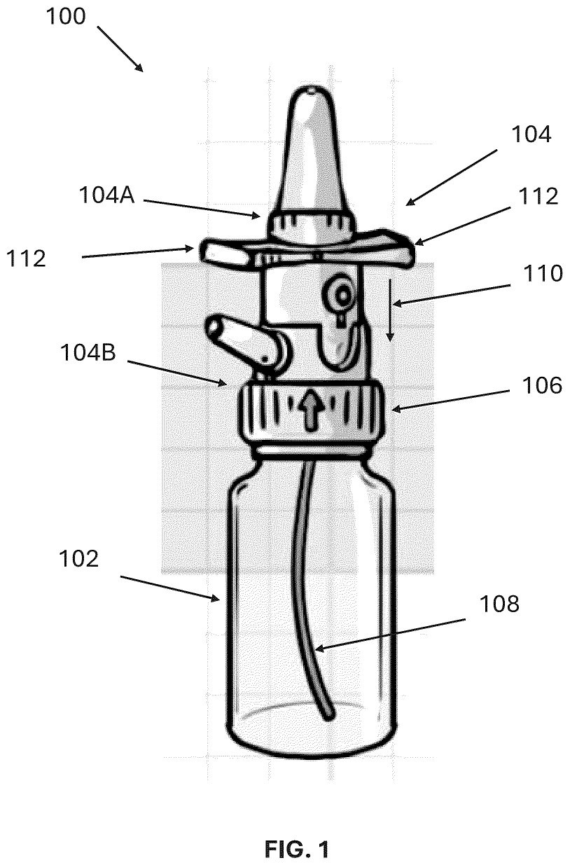

FIG. 1 illustrates a bottle and an applicator of an oronasal conjunctival spray, in accordance with one or more embodiments of the present disclosure. The oronasal conjunctival spray 100 may include a bottle 102 and an applicator 104. The applicator 104 may be screwable to the bottle 102 by a screw 106. The screw 106 may be loosely attached to the applicator 104. The applicator 104 may include a hosepipe 108. Once the applicator 104 is screwed to the bottle 102, the hosepipe 108 extends to the bottom of the bottle 102. The bottle may come in various fluid sizes, such as 30 ml, 60 ml, or 90 ml. The oronasal conjunctival spray can have sizes that are allowable for air travel and portable sizes. The bottle 102 includes glass and/or plastic, which may be transparent/tinted or colored.

The bottle 102 may include a fluid. The fluid may be an isotonic or hyper-tonic saline that can be made at home. The fluid may include a saline solution, prepared by mixing salt and water, enabling the prevention and treatment of viral upper respiratory tract infections. The fluid may include an isotonic or hyper-tonic saline that can be refilled by making the fluid at home. The fluid may include any future antiviral agent that is effective and safe.

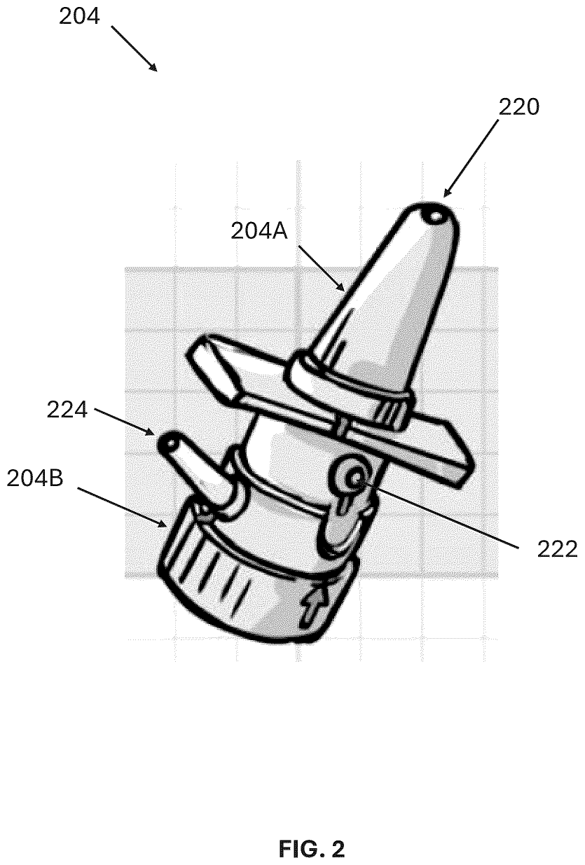

The applicator 104 may include a first part 104A and a second part 104B. The first part 14A may include a cone-shaped tip that is configured to fit inside a nasal cavity. The first part 104A may be inserted into the second part 104B. The first part 104A may be movable against the second part 104B in a direction 110. The applicator 104 may include a spring (not shown) that pushes the first part 104A away from the second part 104B. The applicator 104 may include a lock that locks the first part 104A against the second part 104B at a maximum distance. The first part 104A includes two handles 112 that extend from the first part 104A in opposite directions. A user can place the bottle 102 in the palm of his hand, his index finger on one handle 112 and his middle finger on another handle 112. Then, the user may push the handles 112, using his index and middle finger, towards the second part 104B, which generates high pressure in the applicator 104. This high pressure sprays fluid through one of the ports out of the applicator 102 (see FIG. 2). Once the user release the handles 112, the spring pushes the first part 104A away from the second part 104B until the maximum distance between the first and second part 104A, 104B is reached and the lock prevents the spring to further push the first part 104A away from the second part 104B. The oronasal conjunctival spray 100 may be a portable mechanical device.

FIG. 2 illustrates the applicator of the oronasal conjunctival spray, in accordance with one or more embodiments of the present disclosure.

The applicator 204 of the oronasal conjunctival spray may include a nasal spray port 220, a conjunctival spray port 222, and an oral spray port 224. The nasal spray port 220, a conjunctival spray port 222 may be arranged on the first part 204A of the applicator 204. The oral spray port 224 may be arranged on the second part of the applicator 204. The fluid inside the bottle may exit the applicator 204 through the nasal spray port 220, conjunctival spray port 222, and/or oral spray port 224. The oronasal conjunctival spray may include an adjustment pivot (not shown) that can redirect the fluid from the bottle to one of the nasal spray port 220, conjunctival spray port 222, and/or oral spray port 224. Each of the nasal spray port 220, conjunctival spray port 222, and/or oral spray port 224 may include an orifices (not shown) to adjust the amount of fluid that is sprayed out of the applicator 204. The nasal spray port 220, conjunctival spray port 222, and/or oral spray port 224 may be adjustable to orifices with different sizes for different treatments.

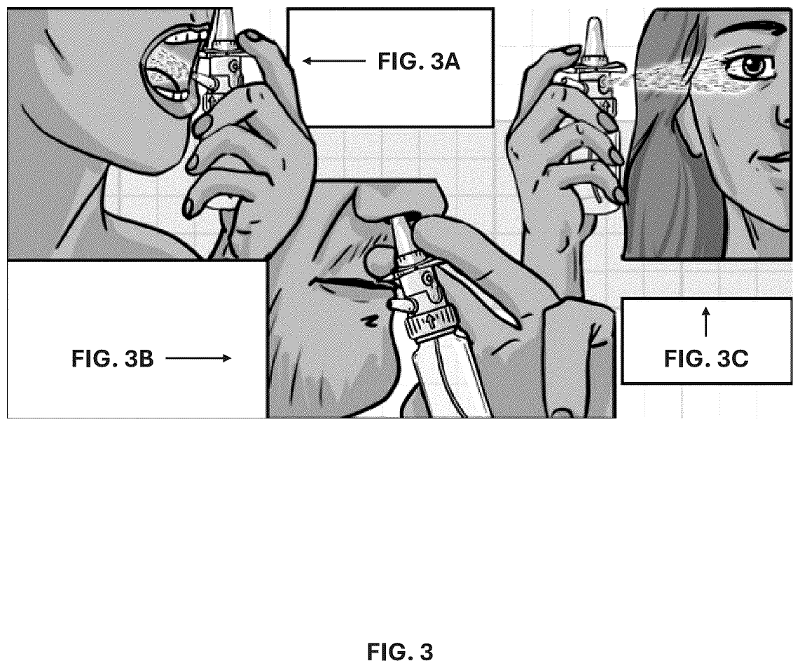

FIGS. 3A, 3B, and 3C illustrate the oronasal conjunctival spray spraying fluid into a mouth, nose, and eye, respectively.

The oronasal conjunctival spray may spray antiviral solution through mouth, nose, and/or eyes. For spraying fluid into a human mouth (see FIG. 3A), the pivot is set to close the nasal spray port and the conjunctival spray port and redirect fluid from the bottle to the oral spray port only. For spraying fluid into a human nose (see FIG. 3B), the pivot is set to close the oral spray port only and the conjunctival spray port and redirect fluid from the bottle to the nasal spray port only. For spraying fluid into a human nose (see FIG. 3C), the pivot is set to close the oral spray port only and the nasal spray port and redirect fluid from the bottle to the conjunctival spray port only.

The oronasal conjunctival spray can be used to prevent and treat viral upper respiratory tract infections (e.g., Covid-19) by acting on the three sites of viral entry (eyes, nose, and mouth) and replication for respiratory viruses. The oronasal conjunctival spray may be used immediately after an exposure or a suspected exposure to a disease. The oronasal conjunctival spray is used to treat early viral infections. The oronasal conjunctival spray can help preventing and treating viral respiratory infections from spreading.

The oronasal conjunctival spray enables healthcare workers, travelers, and home inhabitants, to prevent and treat upper respiratory illnesses at multiple entry points, such as mouth, nose, and/or eyes. The oronasal conjunctival spray is a single device that can be used to clear respiratory viruses attacking humans at all the ports of entry, as well as for early treatment in these infections. The oronasal conjunctival spray is convenient and allows someone to use it immediately after exposure or suspected exposure to viral infections.

What is claimed is:

1. A spray applicator, comprising:
a nasal spray port for spraying fluid into a human nose; and
a conjunctival spray port for spraying fluid into a human eye.

2. The spray applicator of claim 1, further comprising an oral spray port for spraying fluid into a human mouth, which may be gargled.

3. The spray applicator of claim 1, further comprising a pivot to redirect fluid to one of the nasal spray port, conjunctival spray port, or oral spray port.

4. The spray applicator of claim 1, further comprising one or more orifices to adjust an amount of fluid exiting one or more of the nasal spray port, conjunctival spray port, or oral spray port.

5. The spray applicator of claim 1, further comprising a first and second part, wherein the first part is movable against the second part, such that a movement of the first part against the second part sprays fluid from one or more of the nasal spray port, conjunctival spray port, or oral spray port.

6. The spray applicator of claim 5, further comprising a spring that pushes the first and second part away from each other.

7. The spray applicator of claim 5, further comprising a lock that locks the first part against the second part at a maximum distance.

8. A oronasal conjunctival spray, including:
a bottle for fluid; and
a spray applicator comprising:
a nasal spray port for spraying fluid into a human nose, and
a conjunctival spray port for spraying fluid into a human eye, wherein the spray applicator is screwable to the bottle.

9. The oronasal conjunctival spray of claim 8, further comprising a screw that is loosely attached to the spray applicator.

10. The oronasal conjunctival spray of claim 8, wherein the spray applicator includes a hosepipe.
